(19) United States
(12) Patent Application Publication
(10) Pub. No.: US 2010/0255109 A1
(43) Pub. Date: Oct. 7, 2010

(54) STABILIZED ANTIOXIDANT PARTICLES, COMPOSITION COMPRISING THE SAME AND METHOD FOR PREPARING THE SAME

(75) Inventors: Chul Hwan Kim, Daejeon (KR); Chan Jae Shin, Daejeon (KR); So Yeon Seo, Daejeon (KR); Jung Ae Kwon, Daejeon (KR)

Correspondence Address: THE NATH LAW GROUP, 112 South West Street, Alexandria, VA, 22314, US

(73) Assignee: BIOGENICS, INC., Daejeon (KR)

(21) Appl. No.: 12/671,211

(22) PCT Filed: Aug. 4, 2008
(86) PCT No.: PCT/KR08/04508

§ 371 Date: Jan. 29, 2010

(30) Foreign Application Priority Data
Aug. 6, 2007 (KR) 10-2007-0078692
Oct. 30, 2007 (KR) 10-2007-0109312

(51) Int. Cl. A61K 9/14; A61K 31/375; A61K 31/70; A61K 31/353; A61K 36/82; A61P 39/06

(52) U.S. Cl. 424/494; 424/490; 514/474; 514/23; 514/456; 424/729; 424/497; 424/493

(57) ABSTRACT

The present invention provides stabilized antioxidant particles, each of which includes a core consisting of an antioxidant and a first coating layer formed on the surface of the core, wherein the first coating layer is formed by polymerizing at least one α-lipoic acid or its derivative, a composition including the same, and a method for preparing the same.

### TECHNICAL FIELD

The present invention relates to stabilized antioxidant particles, a composition including the same, and a method for preparing the same, and more specifically, to stabilized antioxidant particles, each of which includes a core consisting of an antioxidant and a first coating layer formed on the surface of the core, wherein the first coating layer is formed by polymerizing at least one α-lipoic acid or its derivative, a composition including the same, and a method for preparing the same.

### BACKGROUND ART

Antioxidants such as ascorbic acid (vitamin C) and its derivatives improve immune function in human body, enhance generation of collagen that is an essential constituent of cartilage, capillary vessel, muscle, or the like when applied to the skin, and prevent skin damage by destroying chemicals generated by UV light irradiation. Furthermore, antioxidants retard wrinkles, maintain healthy skin, help repairing damaged skin tissue, and retard aging by inhibiting formation of histamine that is known to cause allergy and formation of melamine that makes skin dark in the process of aging. Thus, the antioxidants have been expected to have excellent functions when applied to not only cosmetic compositions, but also pharmaceutical compositions, food compositions, etc.

However, the antioxidant alone is not stable, particularly in an aqueous medium. Thus, denaturation (e.g., reduction) of the antioxidant may easily occur, and unpleasant odor may also be caused. Ascorbic acid which is widely used as an antioxidant has a structure similar to that of Y-lactone. Due to its structure, ascorbic acid sensitively reacts with environmental factors such as air, particularly oxygen, heat, and light to be easily decomposed. In order to improve stability of ascorbic acid, a method of adding an anti-oxidizing agent, a method of stabilizing ascorbic acid in a multi-lamellar emulsion, a method of stabilizing ascorbic acid in an oil in water type emulsion, and a method of inhibiting oxidization of ascorbic acid using zinc sulfate and L-tyrosine have been reported (U.S. Pat. No. 4,938,969, European Patent Publication No. 533,667 B1, etc.). Furthermore, in order to is improve stability of ascorbic acid, ascorbic acid is chemically modified into a derivative such as sodium ascorbylphosphate, magnesium ascorbyl phosphate, calcium ascorbylphosphate, ascorbic acid polypeptide, ethyl ascorbyl ether, ascorbyl dipalmitate, ascorbyl palmitate, ascorbyl glucoside, and ascorbyl ethylsilanol pectinate.

### DETAILED DESCRIPTION OF THE INVENTION

### Technical Problem

The present inventors conducted various researches in order to develop formulation methods for improving stability of antioxidants, particularly in an aqueous medium. As a result, the present inventors found that, when a coating layer is formed by polymerizing α-lipoic acid or its derivative on the surface of antioxidants, stability of the obtained antioxidant-containing particles is remarkably increased, particularly in an aqueous medium.

Thus, the present invention provides stabilized antioxidant particles, each of which includes a core consisting of an antioxidant and a first coating layer formed by polymerizing α-lipoic acid or its derivative on the surface of the core. The present invention also provides a composition including the stabilized antioxidant particles.

The present invention also provides a method for preparing the stabilized antioxidant particles.

### Technical Solution

According to an aspect of the present invention, there is provided stabilized antioxidant particles, each of which comprises a core consisting of an antioxidant and a first coating layer formed on the surface of the core, wherein the first coating layer is formed by polymerizing at least one α-lipoic acid or its derivative selected from the group consisting of α-lipoic acid, dihydrolipoic acid, α-lipoic acid succinimide, and an amide-bonded compound of α-lipoic acid and amino acid.

According to another aspect of the present invention, there is provided a composition comprising the stabilized α-lipoic acid particles. The composition may be in the form of a food composition, a cosmetic composition, or a pharmaceutical composition.

According to still another aspect of the present invention, there is provided an aqueous cosmetic composition comprising 0.5 to 50% by weight of the stabilized antioxidant particles based on the total weight of the aqueous cosmetic composition.

According to still another aspect of the present invention, there is provided a method for preparing stabilized antioxidant particles, the method comprising: (a) mixing at least one α-lipoic acid or its derivative selected from the group consisting of α-lipoic acid, dihydrolipoic acid, α-lipoic acid succinimide, and an amide-bonded compound of α-lipoic acid and amino acid with an antioxidant to prepare a mixture, and (b) heating the mixture prepared in Step (a) to a temperature of 62 to 100° C., and then cooling the mixture to form a first coating layer.

### ADVANTAGEOUS EFFECTS

The stabilized antioxidant particles according to the present invention include a coating layer formed by polymerizing α-lipoic acid or its derivative, and alternatively an additional water insoluble polymer-coating layer, thereby having excellent stability in an aqueous medium. Thus, the stabilized antioxidant particles can be usefully applied to various compositions including food compositions, pharmaceutical compositions, and cosmetic compositions, since those can minimize denaturation caused by environmental factors such as temperature, light, oxygen, and water, even when being stored in an aqueous composition for a long period of time. Furthermore, the cosmetic compositions including the stabilized antioxidant particles according to the present invention can significantly reduce skin irritation.

### BEST MODE FOR CARRYING OUT THE INVENTION

The present invention provides stabilized antioxidant particles, each of which comprises a core consisting of an antioxidant and a first coating layer formed on the surface of the core, wherein the first coating layer is formed by polymerizing at least one α-lipoic acid or its derivative selected from the group consisting of to α-lipoic acid, dihydrolipoic acid, α-lipoic acid succinimide, and an amide-bonded compound of α-lipoic acid and amino acid.

A variety of conventional antioxidants may be used. For example, the antioxidant may be ascorbic acid, ascorbyl phosphate, glucosyl ascorbate, ascorbyl palmitate, ethyl ascorbyl ether, fructose diphosphate, catechin, epigallocatechin gallate, green tea extract, or the like. The green tea extract may be prepared by extracting green tea leaves using water, a C₁-C₄ alcohol, or a mixture thereof, and includes catechin, polyphenols, etc.

The amount of the antioxidant may be in the range of 50 to 90% by weight, preferably 60 to 80% by weight, based on the total weight of the stabilized antioxidant particles, and the amount of the α-lipoic acid or its derivative may be in the range of 5 to 20% by weight, preferably 5 to 15% by weight, based on the total weight of the stabilized antioxidant particles. If the amount of the α-lipoic acid or its derivative is less than 5% by weight, coating may not be smoothly performed. On the other hand, if the amount of the α-lipoic acid or its derivative is greater than 20% by weight, a polymer of α-lipoic acid-based compound having a high molecular weight may be formed, and thus forming particles may not be easily performed and dissolution of the antioxidant may be delayed. According to an embodiment of the present invention, excellent stability may be obtained by using about 10% by weight of N-α-lipolyl succinimide or about 15% by weight of N-α-lipoyl ethyl glycine.

The stabilized antioxidant particles according to the present invention are obtained by forming a first coating layer through polymerizing α-lipoic acid or its derivative on the surface of a core consisting of the antioxidant. The derivatives of α-lipoic acid may promote polymerization of α-lipoic acid by modifying a carboxylic acid moiety. The modification may be conducted using ethyl glycine, succinimide, or the like. For example, an amide-bonded compound of α-lipoic acid and amino acid refers to an α-lipoic acid derivative (e.g., N-α-lipoyl ethyl glycine) having an amide bond, which can be obtained by coupling α-lipoic acid and glycine or the like, using ethylaminocarboimide, etc.

If desired, the stabilized antioxidant particles may further include a stabilizer having a sulfur atom or a thiol group and/or inorganic particles having an apparent density greater than that of the α-lipoic acid or its derivative. The stabilizer having a sulfur atom or a thiol group may be dimercapto glycerin, cysteine, thiouracil, dithiouracil, C₁-C₂₀ alkyl thio ether, or thioglycolic acid. The inorganic particles may be zeolite, silica, alumina, titania, barium titania, or a mixture thereof. When the first coating layer is formed using the inorganic particles, flowability is increased so that a uniform coating layer may be formed. The inorganic particles may have a nano-sized particle diameter, for example in the range of 5 to 30 nm. The amount of the inorganic particles may be in the range of 1 to 10% by weight based on the total weight of the antioxidant particles, but is not limited thereto.

The first coating layer may be a single layer or a multilayer including at least two layers. For example, the first coating layer may have a single layered structure formed by coating the α-lipoic acid or its derivative, the stabilizer, and the inorganic particles in a single layer. Alternatively, the first coating layer may have a multilayered structure formed by coating α-lipoic acid or its derivative (and optionally stabilizer), and then coating inorganic particles (and optionally stabilizer) as a separate layer. The multilayered structure may be formed by dispersing particles having the coating layer of α-lipoic acid or its derivative, the stabilizer and/or inorganic particles in silicon, etc. and then stirring the dispersion using a Henschel mixer.

The stabilized antioxidant particles according to the present invention may further include a second coating layer formed on the surface of the first coating layer, wherein the second coating layer includes a water insoluble polymer. Any water insoluble polymer that satisfies Korea standard of cosmetic ingredients may be used without limitation. For example, the water insoluble polymer may be at least one selected from the group consisting of polysilane, polysiloxane, polystyrene, poly(methyl methacrylate), cellulose, polycaprolactam, polyacrylic acid, chitosan, and polycaprolactone. The second coating layer may further increase stability of the stabilized antioxidant particles.

The amount of the water insoluble polymer may be in the range of 5 to 20 parts by weight based on 100 parts by weight of α-lipoic acid particles on which the first coating layer is formed. In addition, similarly to the first coating layer, the second coating layer may also further include inorganic particles having an apparent density greater than that of α-lipoic acid or its derivative, for example, zeolite, silica, alumina, titania, and barium titania. The inorganic particles may have a nano-sized particle diameter, for example in the range of 5 to 30 nm. The amount of the inorganic particles may be in the range of 1 to 10% by weight based on the total weight of the antioxidant particles on which the second coating layer is formed, but is not limited thereto. In addition, if desired, the second coating layer may further include a polymer acceptable to the human body, such as polyester (for example, polyester having a weight average molecular weight of about 10,000), and the polymer may make it possible to regulate the amount of the antioxidant in the stabilized antioxidant particles into a desired range.

The second coating layer may be a single layer or a multilayer having at least two layers. For example, the second coating layer may have a single layered structure formed by coating the water insoluble polymer and the inorganic particles in a single layer, or a multilayered structure formed by coating the water insoluble polymer, and then coating the inorganic particles and/or polyester as a separate layer.

The present invention also provides a composition comprising the stabilized antioxidant particles. The composition may be in the form of a food composition, a cosmetic composition, or a pharmaceutical composition. The pharmaceutical composition may comprise a pharmaceutically acceptable carrier. The pharmaceutical composition may be formulated into various forms for oral or external use, such as powders, granules, tablets, capsules, suspensions, emulsions, syrup, and aerosols, in accordance with a conventional method. The pharmaceutically acceptable carrier includes lactose, dextrose, sucrose, sorbitol, mannitol, xylitol, erythritol, maltitol, starch, acacia gum, alginate, gelatin, calcium phosphate, calcium silicate, cellulose, methyl cellulose, amorphous cellulose, poly vinyl pyrrolidone, water, methyl hydroxy benzoate, propyl hydroxy benzoate, talc, magnesium stearate, mineral oil, or the like. In addition, the pharmaceutical composition may include diluents or additives, such as filler, a bulking agent, binder, a wetting agent, disintegrant, surfactant. Solid oral dosage form includes tablets, pills, powders, granules, capsules, or the like. The solid oral dosage form may include at least one additive such as starch, calcium carbonate, sucrose, lactose, and gelatin, and further include a lubricant such as magnesium stearate and talc. Liquid oral dosage form includes suspensions, oral solutions, emulsions, syrup, or the like. The liquid oral dosage form may also include a diluting agent such as water and liquid paraffin, a wetting agent, a sweetener, a fragrance, a preservative, or the like.

A dose of the stabilized antioxidant particles contained in the pharmaceutical composition may vary according to the types of the antioxidant, the status and body weight of patients, degree of disease, dosage form, administration route, and term of administration, and may be appropriately adjusted. For example, the stabilized antioxidant particles may be administered at a dose of 1 to 1000 mg/kg/day, and preferably 1 to 100 mg/kg/day. The stabilized α-lipoic acid particles may be administered once or several times a day. The pharmaceutical composition may be administered to mammals such as human beings via various administration routes such as oral administration, intravenous injection, intramuscular administration, or hypodermic injection.

The composition of the present invention may be in the form of a cosmetic composition comprising the stabilized antioxidant particles as an active ingredient. The cosmetic composition may be prepared using the stabilized antioxidant particles to various forms using a conventional method. For example, the cosmetic composition may be prepared in the form of facial cosmetics, shampoo, hair lotion, hair cream, hair gel, foundation, eye shadow, blusher, nail enamel, eye liner, mascara, lipstick, fancy powder, or the like including the stabilized antioxidant particles, and the cosmetic composition may be diluted using a conventional cleansing solution, astringent, and moisturizer. Furthermore, the cosmetic composition may further include a conventional adjuvant such as stabilizers, solubilizers, vitamins, pigments, and fragrances.

The present invention also provides an aqueous cosmetic composition including 0.5 to 50% by weight of the stabilized antioxidant particles based on the total weight of the aqueous cosmetic composition.

The aqueous cosmetic composition may further include a stabilizer such as glycine, alanine, valine, leucine, isoleucine, threonine, serine, cysteine, methionine, aspartic acid, asparagine, glutamic acid, glutamine, lysine, arginine, histidine, phenylalanine, tyrosine, tryptophan, proline, α-lipoic acid, dihydrolipoic acid, a-lipoic acid succinimide, an amide-bonded compound of α-lipoic acid and amino acid, thioglycolic acid, dimercapto glycerin, cystine, thiouracil, dithiouracil, and C₁-C₂₀ alkyl thio ether, and preferably dihydrolipoic acid or lysine. The amount of the stabilizer may be in the range of 0.01 to 10% by weight based on the total weight of the aqueous cosmetic composition, but is not limited thereto. Specifically, when dihydrolipoic acid is used as the stabilizer, the amount of the dihydrolipoic acid may be in the range of 0.01 to 10% by weight based on the total weight of the aqueous cosmetic composition. When amino acid such as lysine is used as the stabilizer, the amount of the lysine may be in the range of 0.01 to 1% by weight based on the total weight of the aqueous cosmetic composition. In addition, the aqueous cosmetic composition may be in the forms of skin lotion, cream, foundation, or essence.

The present invention also provides a method for preparing stabilized antioxidant particles. The method includes (a) mixing at least one α-lipoic acid or its derivative selected from the group consisting of α-lipoic acid, dihydrolipoic acid, α-lipoic acid succinimide, and an amide-bonded compound of α-lipoic acid and amino acid with an antioxidant to prepare a mixture, and (b) heating the mixture prepared in Step (a) to a temperature of 62 to 100° C., and then cooling the mixture to form a first coating layer.

In the method of the present invention, the first coating layer is formed by heating the mixture including α-lipoic acid or its derivative and the antioxidant to a temperature equal to or higher than 62° C. The melting point of α-lipoic acid is about 61.9° C. When the mixture is heated to a temperature higher than the melting point, S—S bonds of α-lipoic acid are broken, and thus α-lipoic acid is polymerized. The mixture may be heated to a temperature higher than the melting point of α-lipoic acid or its derivative, for example the heating may be performed at about 100° C.

In the method of the present invention, the antioxidant may be at least one selected from the group consisting of ascorbic acid, ascorbyl phosphate, glucosyl ascorbate, ascorbyl palmitate, ethyl ascorbyl ether, fructose diphosphate, catechin, epigallocatechin gallate, and green tea extract. The amount of the antioxidant may be in the range of 50 to 90% by weight, preferably 60 to 80% by weight, based on the total weight of the stabilized antioxidant particles, and the amount of the a-lipoic acid or its derivative may be in the range of 5 to 20% by weight, preferably 5 to 15% by weight, based on the total weight of the stabilized antioxidant particles.

In addition, the mixture prepared in Step (a) may further include a stabilizer having a sulfur atom or a thiol group selected from the group consisting of dimercapto glycerin, cysteine, thiouracil, dithiouracil, C₁-C₂₀ alkyl thio ether, and thioglycolic acid, and/or inorganic particles such as zeolite, silica, alumina, titania, barium titania, or a mixture thereof.

In addition, the method may further include dispersing the stabilized antioxidant particles in a water insoluble polymer or a solution of a water insoluble polymer dissolved in an organic solvent, and then drying the resultant to form a second coating layer. The water insoluble polymer may be at least one selected from the group consisting of polysilane, polysiloxane, polystyrene, poly(methyl methacrylate), cellulose, polycaprolactam, polyacrylic acid, chitosan, and polycaprolactone. When the water insoluble polymer such as polysilane is used, the dispersion may be performed without using the organic solvent. Dichloromethane, tetrahydrofuran, or the like may be used as the organic solvent.

The method may further include adding inorganic particles having an apparent density greater than that of the α-lipoic acid or its derivative after forming the second coating layer, and then stirring the mixture at 100 to 500 rpm. The inorganic particles may be zeolite, silica, alumina, titania, barium titania, or a mixture thereof.

The stabilized antioxidant particles according to the present invention include a coating layer formed by polymerizing α-lipoic acid or its derivative, and alternatively an additional water insoluble polymer-coating layer, thereby having excellent stability in an aqueous medium. Thus, the stabilized antioxidant particles can be usefully applied to various compositions including food compositions, pharmaceutical compositions, and cosmetic compositions, since those can minimize denaturation caused by environmental factors such as temperature, light, oxygen, and water, even when being stored in an aqueous composition for a long period of time.

The present invention will be described in further detail with reference to the following examples. These examples are for illustrative purposes only and are not intended to limit the scope of the present invention.

### Reference Example 1

### Polymerization of α-Lipoic Acid

10 g of α-lipoic acid was melted at 70° C., stirred for 30 minutes, and dried. 50 ml of dichloromethane was added thereto, and the mixture was stirred for 10 minutes and filtered to remove unreacted α-lipoic acid. The resultant was washed three times with acetone to prepare 7.5 g of a polymer of α-lipoic acid (yield 75%).

### Preparation Example 1

### Synthesis of N-α-Lipoyl Succinimide

200 g of α-lipoic acid was dissolved in 2 L of dichloromethane, 1.2 eq. of hydroxy succinimide and 1.2 eq. of pyridine were added to the solution, and the mixture was stirred at room temperature (at about 25° C.) for 10 minutes. A solution, prepared by dissolving 1.2 eq. of ethylaminocarbodiimide in 100 ml of chloroform, was slowly added to the mixture, which was then stirred at room temperature overnight. The reaction mixture was concentrated to obtain a reddish brown product. 3 L of water was added to the product, subjected to extraction using ethyl acetate, and concentrated in a reduced pressure. The resulting residue was crystallized using ether to obtain 250 g of yellow N-α-lipoyl succinimide (yield 80.6%).

m.p. 84-86° C.

¹H NMR 400 MHz ppm 3.59-3.56 (1H, m), 3.22-3.09 (2H, m), 2.83 (4H, s), 2.64-2.58 (2H, t), 2.51-2.43 (1H, m), 1.97-1.90 (1H, m), 1.88-1.70 (2H, m), 1.66-1.61 (2H, m), 1.59-1.50 (2H, m)

### Preparation Example 2

### Synthesis of N-α-Lipoyl Ethyl Glycine

N-α-lipoyl ethyl glycine was prepared in the same manner as in Preparation Example 1, except for using ethyl glycine instead of hydroxy succinimide (yield 78%).

m.p. 63-67° C.

¹H NMR 400 MHz ppm 4.16 (1H, s), 4.17-4.10 (2H, q), 3.60-3.55 (1H, m), 3.24-3.10 (2H, m), 2.66-2.59 (2H, t), 2.52-2.43 (1H, m), 1.98-1.91 (1H, m), 1.89-1.69 (2H, m), 1.67-1.62 (2H, m), 1.59-1.49 (2H, m), 1.32-1.28 (3H, t)

### Examples 1-11

An antioxidant, α-lipoic acid and/or dihydrolipoic acid, and silica were mixed according to the components and amount (g) as shown in Table 1 below. The mixture was placed at 100° C. in an oven for 30 minutes, and then cooled to room temperature. 100 ml of dichloromethane was added thereto, and the mixture was stirred for 10 minutes, filtered, and dried to obtain the coated antioxidant particles (yield: about 80%).

TABLE 1

| | Example | | | | | | | | | | |
|---|---|---|---|---|---|---|---|---|---|---|---|
| | 1 | 2 | 3 | 4 | 5 | 6 | 7 | 8 | 9 | 10 | 11 |
| α-lipoic acid | 20 | 15 | 10 | 20 | 15 | 10 | 20 | 15 | 10 | 15 | 10 |
| Dihydrolipoic acid | — | 5 | 10 | — | 5 | 10 | — | 5 | 10 | 5 | 10 |
| Ascorbic acid | 77.5 | 77.5 | 77.5 | — | — | — | — | — | — | — | — |
| Ascorbyl phosphate | — | — | — | 77.5 | 77.5 | 77.5 | — | — | — | — | — |
| Glucosyl ascorbate | — | — | — | — | — | — | 77.5 | 77.5 | 77.5 | — | — |
| Ascorbyl palmitate | — | — | — | — | — | — | — | — | — | 77.5 | 77.5 |
| Silica | 2.5 | 2.5 | 2.5 | 2.5 | 2.5 | 2.5 | 2.5 | 2.5 | 2.5 | 2.5 | 2.5 |

### Examples 12-22

An antioxidant; α-lipoic acid; dimercapto glycerin, thioglycolic acid, and/or cysteine; and silica were mixed according to the components and amount (g) as shown in Table 2 below. The mixture was placed at 100° C. in an oven for 30 minutes, and cooled to room temperature. 100 ml of dichloromethane was added thereto, and the mixture was stirred for 30 minutes and filtered to obtain the coated antioxidant particles (yield: about 80%).

TABLE 2

| | Example | | | | | | | | | | |
|---|---|---|---|---|---|---|---|---|---|---|---|
| | 12 | 13 | 14 | 15 | 16 | 17 | 18 | 19 | 20 | 21 | 22 |
| α-lipoic acid | 10 | 10 | 10 | 10 | 10 | 10 | 20 | 20 | 20 | 20 | 20 |
| Dimercapto glycerin | 10 | 10 | 5 | — | 10 | 5 | — | — | — | — | — |
| Thioglycolic acid | — | — | — | 5 | — | 5 | — | — | — | — | — |
| Cysteine | — | — | 5 | 10 | — | 5 | — | — | — | — | — |
| Ascorbic acid | 80 | | | | | 75 | | | | | 80 |
| Aascorbyl phosphate | | 80 | | | | | | | | 80 | |
| Glucosyl ascorbate | | | 80 | | | | | | 80 | | |
| Ascorbyl palmitate | | | | 75 | | | | 80 | | | |
| Epigallocatechin gallate | | | | | 80 | | 80 | | | | |
| Silica | 2.5 | 2.5 | 2.5 | 2.5 | 2.5 | 2.5 | 2.5 | 2.5 | 2.5 | 2.5 | 2.5 |

### Examples 23-44

100 parts by weight of the coated antioxidant particles prepared in Examples 1 to 22 were dispersed in 50 parts by weight of dichloromethane, and 10 parts by weight of poly(dimethyl-methylhydrogen)siloxane having a weight average molecular weight of about 10,000 was added thereto. The mixture was stirred at 100 rpm for 10 minutes. The resultant was dried at 60° C. in an oven for 1 hour to obtain the antioxidant particles having a second coating thereon.

### Examples 45-66

100 parts by weight of the coated antioxidant particles prepared in Examples 23 to 44 were mixed with 30 parts by weight of polyester having a weight average molecular weight of about 10,000 and 10 parts by weight of hydrophobic silica. The mixture was uniformly mixed to obtain double-coated antioxidant particles.

### Example 67

### Preparation of Cream

A cream was prepared by mixing 100 g of a cream base shown in Table 3 with 10 g of the coated ascorbic acid particles prepared in Example 45.

TABLE 3

| Cream | Amount [g] |
|---|---|
| Bees wax | 2.0 |
| Stearic acid monoglycerine | 3.0 |
| Liquid paraffin | 4.0 |
| Polysorbate | 5.0 |
| Squalene | 5.0 |
| Hyaluronic acid | 0.3 |
| Glycerine | 4.0 |
| 1,3-butylene glycol | 8.0 |
| Flavoring agent | 0.001 |
| Preservative | 0.3 |
| Distilled water | 68.4 |
| Total | 100.0 |

### Example 68

### Preparation of Cream

A cream was prepared in the same manner as in Example 67, except for using 10 g of the coated ascorbic acid particles prepared in Example 46 instead of the coated ascorbic acid particles prepared in Example 45.

### Example 69

### Preparation of Lotion

A lotion was prepared according to the components and amount (g) as shown in Table 4 below. In Table 4, the coated ascorbic acid particles were prepared in Example 45.

TABLE 4

| Lotion | Amount [g] |
|---|---|
| Stearic acid monoglycerine | 2.5 |
| Liquid paraffin | 4.0 |
| Polysorbate | 1.6 |
| Squalene | 5.0 |
| Carbomer | 0.1 |
| Hyaluronic acid | 0.3 |
| Lycine | 0.3 |
| Glycerine | 4.0 |
| 1,3-butylene glycol | 3.0 |
| Dihydrolipoic acid | 0.2 |
| Coated ascorbic acid particles | 10.0 |
| Flavoring agent | 0.001 |
| Preservative | 0.3 |
| Distilled water | 68.7 |
| Total | 100.0 |

### Example 70

### Preparation of Lotion

A lotion was prepared according to the components and amount (g) as shown in Table 5 below. In Table 5, the coated ascorbic acid particles were prepared in Example 45.

TABLE 5

| Cream | Amount [g] |
|---|---|
| Bees wax | 2.0 |
| Stearic acid monoglycerine | 3.0 |
| Liquid paraffin | 4.0 |
| Polysorbate | 5.0 |
| Squalene | 5.0 |
| Hyaluronic acid | 0.3 |
| Lycine | 0.3 |
| Glycerine | 4.0 |
| 1,3-butylene glycol | 8.0 |
| Dihydrolipoic acid | 0.2 |
| Coated ascorbic acid particles | 10.0 |
| Flavoring agent | 0.001 |
| Preservative | 0.3 |
| Distilled water | 57.9 |
| Total | 100.0 |

### Example 71

A lotion was prepared in the same manner as in Example 69, except that purified water was used instead of lysine and dihydrolipoic acid.

### Comparative Example 1

### Preparation of Cream

A cream was prepared in the same manner as in Example 67, except that ascorbic acid (the same amount of ascorbic acid in the coated ascorbic acid particles prepared in Example 45) was used instead of the coated ascorbic acid particles prepared in Example 45.

### Experimental Example 1

### Measurement of Remaining Ascorbic Acid Amount

Each amount of ascorbic acid in the creams prepared in Examples 67 and 68 and Comparative Example 1 was measured right after preparation and after one month storage at 45° C. The remaining amount of ascorbic acid in the creams was measured using HPLC (Agilent 1200, wavelength: 340 nm), and the results are shown in Table 6. A remaining ratio was calculated using the equation below.

Remaining ratio=amount after one month/amount right after preparation×100

TABLE 6

| | Amount right after | Amount after | Remaining |
|---|---|---|---|
| Cream | preparation | one month | ratio [%] |
| Comparative Example 1 | 98.7 | 23.5 | 23.8 |
| Example 67 | 97.3 | 87.8 | 90.2 |
| Example 68 | 98.3 | 93.7 | 95.3 |
| Example 69 | 97.1 | 94.1 | 96.9 |
| Example 70 | 97.6 | 93.8 | 96.1 |
| Example 71 | 98.1 | 88.1 | 89.8 |

As shown in Table 6, the creams prepared in Examples 67 and 68 showed high ascorbic acid remaining ratio even after one month storage at 45° C. On the other hand, the cream prepared in Comparative Example 1 showed very low remaining ratio after one month storage at 45° C. The lotions prepared in Examples 69 and 70 showed higher ascorbic acid remaining ratio when compared with the creams prepared in Examples 67 and 68 and the lotion prepared in Example 71 which do not include the stabilizer. Thus, it can be seen that dihydrolipoic acid and lysine further increase stability of creams and lotions.

### Experimental Example 2

### Discoloration Test

The creams prepared in Examples 67 and 68 and Comparative Example 1 were stored respectively at 4° C. and 45° C. for 4 weeks, and color density of the creams was measured using GretagMacbeth™ D19C. The results are shown in Table 7. The color density was evaluated in the following standard such that the number is increased as the discoloration progresses: cream base: 0, yellow: 0.5, orange: 0.8, and brown: 2.0. The color density variation (A) is a difference of the color density between at 4° C. and at 45° C.

TABLE 7

| | | | Color |
|---|---|---|---|
| | After 4 weeks | After 4 weeks | density |
| Cream | at 4° C. | at 45° C. | variation (Δ) |
| Comparative Example 1 | 0.33 | 1.59 | 1.26 |
| Example 67 | 0.11 | 0.21 | 0.1 |
| Example 68 | 0.1 | 0.16 | 0.06 |

As shown in Table 7, the color density variation of the creams prepared in Examples 67 and 68 was significantly lower than that of the cream prepared in Comparative Example 1. Thus, the stabilized particles according to the present invention had excellent stability of color density at 45° C.

### Evaluation Example 3

### Skin Irritation Test

Patch tests were performed on the skin of healthy people using creams prepared in Examples 67 and 68 and Comparative Example 1. Test patches were attached to the skin every 24 hours and the degree of skin irritation was measured for 3 days. The results are shown in Table 8. The degree of the skin irritation was classified into 0-5 levels as shown below.

0: no redness and no irritation

1: no redness and very light irritation

2: faint redness and light irritation

3: distinct redness and light irritation

4: distinct redness and light pain

5: distinct redness and strong pain

TABLE 8

| Cream | Degree of Skin Irritation |
|---|---|
| Comparative Example 1 | 3.5 |
| Example 67 | 0.5 |
| Example 68 | 0.5 |

As shown in Table 8, skin irritation of the creams prepared in Examples 67 and 68 was significantly less than that of the cream prepared in Comparative Example 1. It can be assumed that the multi-coated antioxidant was remained in a very stable state in the creams and gradually eluted over time, and thus skin irritation was alleviated.

### Evaluation Example 4

### Sensory Test (Odor)

5% by weight of the stabilized antioxidant particles prepared in Examples 1 to 22 were added to an aqueous cream having the composition shown in Table 3, and the cream was stored at 45° C. for 1 month. Then, sensory test (odor) for the cream was performed. As a result, the aqueous cream including the stabilized antioxidant particles of Examples 1 to 22 did not have any odor. For comparison, a sensory test for the cream prepared by using ascorbic acid was performed in the same manner as described above, and the cream had an unpleasant odor.

1. Stabilized antioxidant particles, each of which comprises a core consisting of an antioxidant and a first coating layer formed on the surface of the core, wherein the first coating layer is formed by polymerizing at least one α-lipoic acid or its derivative selected from the group consisting of α-lipoic acid, dihydrolipoic acid, α-lipoic acid succinimide, and an amide-bonded compound of α-lipoic acid and amino acid.

2. The stabilized antioxidant particles of claim 1, wherein the antioxidant is at least one selected from the group consisting of ascorbic acid, ascorbyl phosphate, glucosyl ascorbate, ascorbyl palmitate, ethyl ascorbyl ether, fructose diphosphate, catechin, epigallocatechin gallate, and green tea extract.

3. The stabilized antioxidant particles of claim 1, wherein the amount of the antioxidant is in the range of 50 to 90% by weight based on the total weight of the stabilized antioxidant particles, and the amount of the α-lipoic acid or its derivative is in the range of 5 to 20% by weight based on the total weight of the stabilized antioxidant particles.

4. The stabilized antioxidant particles of claim 1, wherein the first coating layer further comprises a stabilizer having a sulfur atom or a thiol group selected from the group consisting of dimercapto glycerin, cysteine, thiouracil, dithiouracil, C₁-C₂₀ alkyl thio ether, and thioglycolic acid.

5. The stabilized antioxidant particles of claim 1, wherein the first coating layer further comprises inorganic particles having an apparent density greater than that of the α-lipoic acid or its derivative.

6. The stabilized antioxidant particles of claim 5, wherein the inorganic particles are selected from the group consisting of zeolite, silica, alumina, titania, barium titania, ands a mixture thereof.

7. The stabilized antioxidant particles of claim 1, further comprising a second coating layer formed on the surface of the first coating layer, wherein the second coating layer comprises a water insoluble polymer.

8. The stabilized antioxidant particles of claim 7, wherein the water insoluble polymer is at least one selected from the group consisting of polysilane, polysiloxane, polystyrene, poly(methyl methacrylate), cellulose, polycaprolactam, polyacrylic acid, chitosan, and polycaprolactone.

9. The stabilized antioxidant particles of claim 8, wherein the second coating layer further comprises inorganic particles having an apparent density greater than that of the α-lipoic acid or its derivative.

10. The stabilized antioxidant particles of claim 9, wherein the inorganic particles are selected from the group consisting of zeolite, silica, alumina, titania, barium titania, and a mixture thereof.

11. A composition comprising the stabilized antioxidant particles according to claim 1.

12. The composition of claim 11, being a food composition, a cosmetic composition, or a pharmaceutical composition.

13. An aqueous cosmetic composition comprising 0.5 to 50% by weight of the stabilized antioxidant particles according to claim 1 based on the total weight of the aqueous cosmetic composition.

14. The aqueous cosmetic composition of claim 13, further comprising at least one stabilizer selected from the group consisting of glycine, alanine, valine, leucine, isoleucine, threonine, serine, cysteine, methionine, aspartic acid, asparagine, glutamic acid, glutamine, lysine, arginine, histidine, phenylalanine, tyrosine, tryptophan, proline, α-lipoic acid, dihydrolipoic acid, α-lipoic acid succinimide, an amide-bonded compound of α-lipoic acid and amino acid, thioglycolic acid, dimercapto glycerin, cystine, thiouracil, dithiouracil, and C₁-C₂₀ alkyl thio ether.

15. The aqueous cosmetic composition of claim 14, wherein the stabilizer is dihydrolipoic acid or lysine.

16. The aqueous cosmetic composition of claim 14, wherein the amount of the stabilizer is in the range of 0.01 to 10% by weight based on the total weight of the aqueous cosmetic composition.

17. The aqueous cosmetic composition of claim 13, in the form of skin lotion, cream, foundation, or essence.

18. A method for preparing stabilized antioxidant particles, the method comprising:
(a) mixing at least one α-lipoic acid or its derivative selected from the group consisting of α-lipoic acid, dihydrolipoic acid, α-lipoic acid succinimide, and an amide-bonded compound of α-lipoic acid and amino acid, with an antioxidant to prepare a mixture, and
(b) heating the mixture prepared in Step (a) to a temperature of 62 to 100° C., and then cooling the mixture to form a first coating layer.

19. The method of claim 18, wherein the antioxidant is at least one selected from the group consisting of ascorbic acid, ascorbyl phosphate, glucosyl ascorbate, ascorbyl palmitate, ethyl ascorbyl ether, fructose diphosphate, catechin, epigallocatechin gallate, and green tea extract.

20. The method of claim 18, wherein the amount of the antioxidant is in the range of 50 to 90% by weight based on the total weight of the stabilized antioxidant particles, and the amount of the α-lipoic acid or its derivative is in the range of 5 to 20% by weight based on the total weight of the stabilized antioxidant particles.

21. The method of claim 18, wherein the mixture prepared in Step (a) further comprises a stabilizer having a sulfur atom or a thiol group selected from the group consisting of dimercapto glycerin, cysteine, thiouracil, dithiouracil, C₁-C₂₀ alkyl thio ether, and thioglycolic acid.

22. The method of claim 18, wherein the mixture prepared in Step (a) further comprises inorganic particles having an apparent density greater than that of the α-lipoic acid or its derivative.

23. The method of claim 22, wherein the inorganic particles are selected from the group consisting of zeolite, silica, alumina, titania, barium titania, and a mixture thereof.

24. The method of claim 18, further comprising dispersing the stabilized antioxidant particles prepared according to claim 18 in a water insoluble polymer or a solution of a water insoluble polymer dissolved in an organic solvent, and then drying the resultant to form a second coating layer.

25. The method of claim 24, wherein the water insoluble polymer is at least one selected from the group consisting of polysilane, polysiloxane, polystyrene, poly(methyl methacrylate), cellulose, polycaprolactam, polyacrylic acid, chitosan, and polycaprolactone.

26. The method of claim 24, further comprising adding inorganic particles having an apparent density greater than that of the α-lipoic acid or its derivative after forming the second coating layer; and then stirring the mixture at 100 to 500 rpm.

27. The method of claim 26, wherein the inorganic particles are selected from the group consisting of zeolite, silica, alumina, titania, barium titania, and a mixture thereof.
